(19) United States
(12) Patent Application Publication
(10) Pub. No.: US 2010/0178255 A1
(43) Pub. Date: Jul. 15, 2010

(54) Composition and method to treat otitis media and ventilator- associated pneumonia

(76) Inventors: Madhavi Sekharam Kotha, Tampa, FL (US); Anupama Kotha, Tampa, FL (US)

Correspondence Address: MADHAVI S. KOTHA, 9702 TREE TOPS LAKE ROAD, TAMPA, FL, 33626, US

(21) Appl. No.: 12/321,066

(22) Filed: Jan. 15, 2009

(51) Int. Cl. A61K 9/12; A61K 31/352; A61P 31/00

(52) U.S. Cl. 424/45; 514/456

(57) ABSTRACT

A composition and method for prevention and treatment of otitis media and ventilator-associated pneumonia are described. The composition is made with GTase inhibitory polyphenol and polyhydric alcohol carrier. The composition is topical for intra-oral and otic use.

### FIELD OF THE INVENTION

This invention relates to means of preventing and treating otitis media and ventilator-associated pneumonia in mammals, including humans. The invention further relates to a pharmaceutical composition and method comprising GTase inhibitory polyphenol and polyhydric alcohol carrier, including xylitol. The invention is topical in use with intra-oral and otic applications and hence no oral ingestion is needed.

### SUMMARY OF THE INVENTION

The present invention relates to a composition intended for the use by patients with otitis media and also for patients with potential risk for ventilator-associated pneumonia. The composition includes GTase inhibitory polyphenol and polyhydric alcohol carrier. The composition is a solution, suspension, gel, foam, cream, solid, aerosol, or a suitable form to convey therapeutically effective components to the target area.

### BACKGROUND OF THE INVENTION

The present invention relates to composition and method for prevention and treatment of otitis media and ventilator-associated pneumonia. The composition is made with GTase inhibitory polyphenol and polyhydric alcohol carrier.

Otitis media (OM) is an infection or inflammation of the middle ear. OM is classified as acute otitis media (AOM), or as otitis media with effusion (OME), a chronic disease. It is the most prevalent infectious disease affecting young children, and the major cause of conductive hearing loss among this group. More than one third of children experience 6 or more episodes of AOM by age 7 years. This is also common in animals, including dogs and cats.

Otitis media results in 31 million annual visits to physicians' offices and is estimated to have a yearly cost exceeding $5 billion. Most cases of OM are caused by viral or bacterial infections.

Bacteria and viruses can cause otitis media. Bacteria such as Streptococcus pneumonia (pneumococcus), non-typeable Hemophilus influenza, and Moraxella account for about 85% of cases of acute otitis media. Viruses account for the remaining 15%.

Acute otitis media is treated with antibiotics and acetaminophen. First line treatment antibiotic is still Amoxicillin in an appropriate dose. Alternative second line antibiotics include amoxicillin-clavulanic acid; cephalosporins such as cefaclor, cefixime, cefuroxime axetil, and cefpodoxime, macrolides such as azithromycin and clarithromycin; and sulfa drugs such as trimethaprim-sulfamethoxazole and erythromycin-sulfisoxazole.

Ventilator-associated pneumonia (VAP) is defined as pneumonia (infection of the lung) occurring to a person who has been assisted by mechanical ventilation within the past 48 hours. During VAP, endotracheal tube allows free passage of bacteria into lower segment of the lung and this may happen within the first 48 hours after intubation.

Despite advanced management techniques and disinfestation procedures for equipment, VAP complicates the course of 8 to 28% of patients receiving mechanical ventilation. Since the VAP patient is already critically ill, mortality rate is very high, ranging from 24 to 50% and can reach 76% in some specific settings. ICU ventilated patients with VAP appear to have a 2 to 10-fold higher risk of death compared to patients without pneumonia. Death rates associated with Pseudomonas pneumonia are particularly high, ranging from 70 to more than 80% in several studies.

Several studies have reported that more than 60% of VAP is caused by aerobic gram negative bacteria (GNB). Gram-positive bacteria, including S. aureus is also common. In sicker patients who have been hospitalized more than 5 days, GNB and S. aureus frequently colonize the upper airway. Prolonged antibiotic administration to ICU patients for primary infection is thought to favor selection and subsequent colonization of resistant pathogens responsible for super infections.

Core organisms responsible for VAP include Enterobacter spp., Escherichia coli Klebsiella spp., Proteus spp., Serratia marcescens, Haemophilus influenzae, Streptococcus pneumonia, Pseudomonas aeruginosa, and Acinetobacter baumanni, of which Streptococcus pneumoniae (S. pneumoniae) (30-40%), non-typeable Haemophilus influenzae (NTHi) (30%) and Moraxella catarrhalis (M. catarrhalis) (20%) are more prominent.

There are about 250,000 patients with VAP. The extra hospital charges attributed to nosocomial pneumonia occurring in trauma patients were evaluated to be $40,000. Hospital cost per infection (year 2002) is estimated to be $9,969.

About 70% of bacteria that cause infections in hospitals are resistant to at least one of the drugs most commonly used to treat infections. Since some organisms are resistant to all approved antibiotics, the only choice is to treat with experimental and potentially toxic drugs. Resistance of Streptococcus pneumoniae to penicillin and other beta-lactams is increasing worldwide. Streptococcus pneumoniae is responsible for pneumonia, bacteremia, otitis media, meningitis, sinusitis, peritonitis, and arthritis.

In view of the increasing antibiotic resistance, there is a pressing need to develop alternate or additional methods, preferably without using antibiotics to control spread of oral and otic infectious microorganisms.

Polyphenols are a group of chemical substances found in plants. They are characterized by the presence of more than one phenol unit or building block per molecule. It has been reported that certain natural polyphenols inhibit the GTase-I and thus prevent adherence of microorganism to teeth and solid surfaces in the oral area (K Nakahara, S Kawabata, H Ono, K Ogura, T Tanaka, Ooshima, and S Hamada, Inhibitory effect of oolong tea polyphenols on glycosyltransferases of mutans Streptococci. Appl. Environ Microbiol. April 1993; 59(4): 968-973).

Polyphenols are also known to control oral bacteria. U.S. Pat. No. 6,319,523 teaches polyphenol composition to inhibit oral bacteria. U.S. Pat. No. 6,159,447 teaches a composition with polyphenol for controlling bacterial colonization. U.S. Pat. No. 5,204,089 teaches a method of preventing the formation or aggravation of dental plaque with at least one tea polyphenol.

Topical use of polyphenols is well documented. Polyphenols are used topically for conditioning of skin (U.S. Pat. No. 7,314,634), for peripheral neural and vascular ailments (U.S. Pat. No. 7,083,813), for cosmetic purpose (U.S. Pat. No. 6,508,411), as a topical antioxidant (U.S. Pat. No. 6,146,616), and for skin peel composition (U.S. Pat. No. 5,874,463).

There is no prior knowledge on topical use of polyphenols, particularly GTase inhibitory polyphenols to treat either otitis media or ventilator-associated pneumonia.

Xylitol is a naturally occurring sugar alcohol. Antimicrobial and antiadhesive properties of orally ingested xylitol have been established [(1) Matti Uhari et. al; A Novel Use of Xylitol Sugar in Preventing Acute Otitis Media. Pediatrics 102(4) 1998: 879-884 (2) Terhi Tapiaine et. al; Effect of Xylitol on Growth of Streptococcus pneumoniae in the Presence of Fructose and Sorbitol. Antimicrobial Agents and Chemotherapy, 2001, 45 (1):166-169 (3) T. Tapiainen et. al; Xylitol Concentrations in the Saliva of Children After Chewing Xylitol Gum or Consuming a Xylitol Mixture, European Journal of Clinical Microbiology & Infectious Diseases, 21(1) 2002; (4) T Kontiokari, et. al; Antiadhesive effects of xylitol on otopathogenic bacteria, Jr. of Antimicrobiol. Chemotherapy, 41, 563-565, 1998; (5) Terhi Tapiainen et. al; Ultrastructure of Streptococcus pneumoniae after exposure to xylitol, J. Antimicrob. Chemother., July 2004; 54: 225-228 (6) T Kontiokari et. al; Effect of xylitol on growth of nasopharyngeal bacteria in vitro, Antimicrobial Agents and Chemotherapy, 1820-1823 (39),1995].

U.S. Pat. No. 5,719,196 and U.S. Pat. No. 6,066,677 teach method of treating respiratory infections with oral ingestion of liquid, solid preparations of xylitol in the form of chewing gum etc.

There is no prior knowledge on topical (involving no oral ingestion) use of xylitol, particularly in conjunction with polyphenols to treat either otitis media or ventilator-associated pneumonia.

Now it has been discovered that GTase inhibitory polyphenol and polyhydric alcohol carrier such as xylitol is combined to prevent or treat otitis media and ventilator-associated pneumonia.

It is also discovered that the present composition is therapeutically effective without a need for oral ingestion. This is very crucial for patients connected with ventilator, as they may not be in a position to swallow any material.

### BRIEF DESCRIPTION OF THE DRAWINGS

Some of the features, advantages, and benefits of the present invention have been stated, and others will become apparent as the description proceeds when taken in conjunction with the accompanying data in tables presented solely for exemplary purpose-and not with the intent to limit the invention hereto.

TABLE 1 Composition examples

TABLE 2 Minimum Inhibitor Concentration (MIC) of the Test Composition

### DETAILED DESCRIPTION OF THE PREFERRED EMBODIMENT

The present composition constitutes (a) GTase inhibitory polyphenol (b) Polyhydric alcohol carrier and (c) optional antimicrobial. The composition is liquid, solution, suspension, gel, spray, cream, solid, aerosol, or a lotion intended for topical intra-oral or otic applications for mammals, including humans with otitis media, ear infections and also for those with potential risk for ventilator-associated pneumonia.

The term “GTase inhibitory polyphenol” is a polyphenol that can inhibit GTase enzyme activity of the infectious microorganisms. Certain oral microorganisms, including Streptococcus, synthesize extracellular water-soluble glucans by using glucosyltransferase I (GTase-I) enzyme. These glucans are highly adherent to solid surfaces, including teeth surface.

The GTase inhibitory polyphenol can vary and can include one or more of epigallocatechin gallate, epigallocatechin, epicatechin gallate, epicatechin, gallocatechin gallate, gallocatechin, catechin gallate, and catechin. Each of catechins described here may be either (+)-form or (−)-form, and (−)-EGCg, (−)-EGC, (−)-ECg, (−)-EC, (−)-GCg, (−)-GC, (−)-Cg and (+)-C are preferable. These compounds are abundant in teas derived from the tea-plant Camellia sinensis as well as in some cocoas and chocolates (made from the seeds of Theobroma cacao). One cup (240 mL) of brewed green tea contains up to 200 mg EGCg. With centuries of human use, these compounds are proved to be safe for consumption.

Polyphenols can be prepared from the raw material-tea leaves, and a method for the-preparation thereof is described in U.S. Pat. Nos.4,613,672, and 4,673,530. Polyphenols are commercially available from MITSUI NORIN CO., LTD. (trade name: Polyphenon™) and TAIYO KAGAKU CO., LTD. (trade name: Sunphenon™).

Either purified polyphenol or botanicals containing polyphenols may also be used as a source. Some of the non-limiting examples are Quercetin, Gingerol, Kaempferol, Myricetin, Resveratrol, Rutin, Isorhamnetin, Hesperidin, Naringenin, Silybin, Eriodictyol, Apigenin, Tangeritin, Luteolin, Thearubigins, Pelargonidin, Peonidin, Cyanidin, Delphinidin, Malvidin, Petunidin, Daidzein, Genistein, Glycitein, Coumestans, Coumestrol, Ellagic acid, Gallic acid, Tannic acid, Curcumin, Caffeic acid, Chlorogenic acid, Cinnamic acid, Ferulic acid, Coumarin, Silymarin, Matairesinol, Secoisolariciresinol, Pinoresinol, Lariciresinol, Tyrosol, Hydroxytyrosol, Oleocanthal, Oleuropein, Pterostilbene, and Piceatannol. One or more polyphenols can be used in the composition.

Polyphenols in the composition may range from about 0.01% to about 50% w/w with a preferable usage from about 0.5% to about 10% w/w of the final composition.

The composition also includes a polyhydric alcohol (also called polyols) carrier. The term “polyhydric alcohol” refers to alcohols containing multiple hydroxyl groups. Polyhydric alcohols are not metabolized by oral bacteria and are therefore not acidogenic or cariogenic. Some examples of polyols include erythritol, maltitol, mannitol, sorbitol, xylitol, lactitol, isomalt, and hydrogenated starch hydrolyzate. The polyhydric alcohol serves the function of a vehicle to carry polyphenol component of the composition. Xylitol is the prefereed carrier. Xylitol is non-cariogenic, provides same sweetness and taste as sugar, and possesses antimicrobial properties. Xylitol is commercially available from, various sources, for example as Xylisorb® brand from ROQUETTE.

Polyhydric alcohol in the composition may range from about 1% to about 90% W/W of the final composition. Usage levels of xylitol depend on the presence of other ingredients, desired viscosity, and desired physical properties of the final product. Preferable usage levels are from about 20% to about 60% w/w of the final composition.

The present composition can also include optional antimicrobial agents. Antimicrobial agents are natural or chemical agent that can kill or inhibit the growth of microorganisms. Penicillins (Amoxicillin, Amoxicillin-clavulanate), sulfa-based combinations (Erythromycin-sulfisoxazole, Trimethoprim-sulfamethoxazole), Macrolide/azalid (Azithromycin, Clarithromycin), second-generation cephalosporins (Cefaclor, Cefprozil, Cefuroxime), third-generation cephalosporins (Cefdinir, Cefixime, Cefpodoxime proxetil, Ceftibuten, Ceftriaxone), antimicrobials such as quinolones and silver sulfadiazine and the like can be included in the composition.

Natural products containing antimicrobial properties can also be included in the present composition. Some non-limiting botanical examples are turmeric, curcumin, neem, and ocimum. Other botanicals with similar antimicrobial properties can also be included in the composition. Honey is another example of natural product with proven antimicrobial property and it can also be included in the composition.

The present composition can also include inactive ingredients like water, alcohol, thickeners including carbomer, neutralizing agents like TEA, preservatives, penetration-enhancers, emulsifiers, stabilizers, antioxidants, colors, flavors, taste modifiers, and surfactants that may help achieve required consistency, viscosity, flow properties, adhesive properties, and the like.

The composition can be liquid, solution, suspension, paste, gel, spray, aerosol, and the forms known to the trade. The composition can also be a solid that dissolves in the target area due to saliva or moisture.

The composition can be applied intra-orally by several methods, for example, it can be sprayed, rubbed with an applicator and gargled. For otitis media, the composition can be dropped in to ear canal. The present composition's application is topical-inside the oral area and in the ear. To achieve the pharmacological efficacy of the composition, there is no need to ingest the composition. However, the composition is safe for ingestion, as it is made with natural constituents.

The following non-limiting examples further demonstrate method of preparation and application of pharmaceutical compositions according to the invention. Unless otherwise noted, all parts and percentages are by weight.

### Manufacture Process

Dissolve polyphenol in alcohol. Sieve in carbomer in water and disperse. Dissolve preservatives. Dissolve powder xylitol in water and combine with polyphenol-alcohol solution. Check and adjust pH to desired level (4 to 7 depending on the end use).

The composition prepared according to the method described is suitable for packaging in dropper bottles, spray bottles, or other customary pharmaceutical containers. The composition can be modified to suit other needs: otic solution can be prepared with pH as low as 4, topical gel can be prepared with high viscosity, mucoadhesive composition can be prepared for intra-oral application, composition can also be prepared with or without alcohol, etc.

### Proof

Efficacy of the composition was determined by the MIC (minimum. inhibitory concentration) assay against selected microorganism using the broth macro-dilution procedure for antibiotic susceptibility testing (National Committee for Clinical Laboratory Standards, NCCLS). The inoculum for each microorganism is prepared and the MIC assay conducted according to respective culture methods described by NCCLS (M7-A5,2000). The following microorganisms are tested: Streptococcus pneumoniae ATCC # BAA-343 (resistant to multiple antibiotics including amoxicillin and penicillin), Haemophilus influenza ATCC # 33929 (resistant to multiple antibiotics including ampicillin), and Moraxella catarrhalis ATCC #25238. Test results indicate the efficacy of the present composition.

Although preferred embodiments of the invention have been described herein detail, it will be understood by those skilled in the art that variations may be made hereto without departing from the spirit of the invention.

TABLE 1
Composition Examples (% w/w)

| | | Formula-1 | Formula-2 |
|---|---|---|---|
| | | (Spay-gel for | (Solution for otic |
| | | intra-oral use) | application) |
| 1 | Polyphenol | 1 | 2.5 |
| | (Sunphenon TM | | |
| | EGCg 90%) | | |
| 2 | Alcohol (ethanol) | 15 | 10 |
| 3 | Xylitol | 30 | 43.5 |
| 4 | Carbopol | 0.3 | 0.2 |
| 5 | Methyl paraben | 0.02 | 0.02 |
| 6 | Propyl paraben | 0.01 | 0.01 |
| 7 | Antimicrobial | 0 | 0 |
| 8 | Water | to 100 | to 100 |

TABLE 2
MIC of the Test Composition

| | | Results (turbidity/growth) | | |
|---|---|---|---|---|
| | Test Material | Streptococcus | Moraxella | Haemophilus |
| | Dilution | pneumoniae | catarrhalis | influenzae |
| 1 | 0.1 | − | − | − |
| 2 | 0.05 | + | − | − |
| 3 | 0.025 | + | − | + |
| 4 | 0.0125 | + | − | + |
| 5 | 0.00625 | + | − | + |
| 6 | 0.003125 | + | + | + |

Test material was diluted 1:10 in the appropriate growth media. The precipitate was removed via centrifugation/filtration and the clarified solution used in the MIC series above.

What is claimed is:

1. A composition for prevention and treatment of otitis media and ventilator-associated pneumonia comprising:
a. GTase inhibitory polyphenol
b. Polyhydric alcohol carrier
c. Optional antimicrobial
d. and therapeutically effective amount of such composition is administered to mammals, including humans in need of such treatment.

2. The composition of claim 1, wherein GTase inhibitory polyphenol is selected from the group consisting of epicatechin gallate, gallocatechin gallate, epigallocatechin gallate, epicatechin, epigallocatechin, gallocatechin, catechin gallate, catechin, free theaflavin, theaflavin monogallate A, theaflavin monogallante B and theaflavin digallate, botanicals containing such polypehnols and a combination thereof.

3. The composition of claim 1, wherein the carrier is selected from the group consisting of erythritol, maltitol, mannitol, sorbitol, xylitol, lactitol, isomalt, hydrogenated starch hydrolysate, xylitol, and a combination thereof.

4. The composition of claim 1, wherein the carrier is xylitol.

5. The composition of claim 1, wherein the GTase inhibitory polyphenol is about 0.01% to about 50% w/w of the final composition.

6. The composition of claim 1, wherein the GTase inhibitory polyphenol is about 0.5% to about 20% w/w of the final composition.

7. The composition of claim 1, wherein the GTase inhibitory polyphenol is about 0.5% to about 10% w/w of the final composition.

8. The composition of claim 1, wherein the polyhydric alcohol is about 1% to about 90% w/w of the final composition.

9. The composition of claim 1, wherein the polyhydric alcohol is about 10% to about 70% w/w of the final composition.

10. The composition of claim 1, wherein the polyhydric alcohol is about 20% to about 60% w/w of the final composition.

11. The composition of claim 1 is solution, suspension, gel, foam, cream, solid, and aerosol.

12. The composition of claim 1 optionally comprising one or more antimicrobial ingredients.

13. The composition of claim 1 is administered topically in intra-oral area.

14. The composition of claim 1 is administered topically in intra-otic area.

15. A method of preventing otitis media comprising administering therapeutically effective amount of the composition of claim 1, to mammals including humans in need of such treatment.

16. A method of treating otitis media comprising administering therapeutically effective amount of the composition of claim 1, to mammals including humans in need of such treatment.

17. A method of treating, preventing otic infections comprising administering therapeutically effective amount of the composition of claim 1, to mammals including humans in need of such treatment.

18. A method of preventing ventilator-associated pneumonia comprising administering therapeutically effective amount of the composition of claim 1, to mammals including humans in need of such treatment.

19. A method of treating ventilator-associated pneumonia comprising administering therapeutically effective amount of the composition of claim 1, to mammals including humans in need of such treatment.
